(19) United States
(12) Patent Application Publication
(10) Pub. No.: US 2005/0153841 A1
(43) Pub. Date: Jul. 14, 2005

(54) Injection formulation

(76) Inventors: Craig Robert Bunt, Hamilton (NZ); Colin Roger Ogle, Hamilton (NZ); Michael John Rathbone, Hamilton (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/990,089

(22) Filed: Nov. 15, 2004

Related U.S. Application Data
(63) Continuation of application No. PCT/NZ03/00095, filed on May 16, 2003.

(30) Foreign Application Priority Data
May 16, 2002 (NZ) 518997

(51) Int. Cl. A01N 25/04; A01N 25/16; A61K 47/30

(52) U.S. Cl. 504/366; 514/772.3

(57) ABSTRACT

The present invention relates to a formulation for parenteral consisting of a gelling agent or agents, a water miscible solvents and an active agent or agents. The aim of the suspension is to deliver active agent or agents to a subject in the form of a depot that, on administration to the subject, forms a depot. The depot will disperse in time and during this time period, active agent or agents are released into the subject.

### CROSS-REFERENCE TO RELATED APPLICATIONS

This application is a continuation of and claims priority under 35 U.S.C. § 120 to PCT Application No. PCT/NZ03/00095, filed on May 16, 2003 and published in English on Nov. 27, 2003, entitled INJECTION FORMULATION; which application claimed priority to New Zealand Application No. 518997, which was filed on May 16, 2002; each of which applications is incorporated herein by reference in its entirety.

### BACKGROUND OF THE INVENTION

1. Field of the Invention

The invention relates to a formulation administered parenterally to a subject.

More specifically the invention relates to a particulate formulation, which, on administration to a subject such as an animal, forms a depot.

2. Description of the Related Art

In modern medicine it is common to treat a subject by administration of an active agent. The agent has a variety of effects including, for example, releasing an antibiotic into the subject's bloodstream.

Typical methods of administration of such agents are often tablets, capsules, injections and other such methods. A method also employed is the use of an implant containing an active ingredient.

Implants are particularly advantageous as the active agent can be released over a time period whereas other treatments such as tablets breakdown rapidly once administered to the subject. In the case of animal medicine, implants are a desirable means of treating an animal as the treatment need only be applied once and the animal is then treated on a progressive basis without further intervention by the vet or animal handler.

A typical implant is often a device which is either expelled from the animal once administration has completed or the implant breaks down in the animal and is expelled during normal bodily functions.

While the implant device has obvious advantages as described above, they also have problems with regard to the method of administration and the type of materials used.

Some implant devices do not breakdown and are either removed surgically or not removed at all. This can cause problems to the subject such as leaching of toxic materials from the implant, a physical lump or obstruction in the body of the subject, and in the case of farm animals, solid objects on which machinery used for processing the animal can become caught or damaged by.

It is therefore desirable to have an implant that is a biocompatible material to the subject and which can break down within the subject.

Implants, being solid or semi-solid devices, are difficult to administer to internal regions of the subject. The devices have traditionally been administered either orally, physically inserted into a body cavity, or inserted surgically.

Each of these methods has their limitations.

Oral and physical insertion limits the location of the device to the oral passage and/or body cavities. Surgery can insert these devices into areas oral and physical methods cannot reach, however this method is traumatic for the subject, expensive and time consuming. As a result it is only warranted in extreme circumstances.

Other methods of administering the device to a subject are difficult or impossible, for example, injection formulations need to be of low to moderate viscosity in order to operate the injection device e.g. plunge the syringe. Implants thus cannot be administered via injection due to the solid/semi-solid nature of the device.

It is therefore desirable to have a method of administration of an implant that can be administered to a subject easily, cheaply and preferably into locations within the subject that cannot be reached by alternative methods e.g. the oral route.

An alternative method used to address the above problems regarding implant administration has been to use solvents for carrying the implant material where the solvent/implant material is injected into the subject. Typical examples are described in U.S. Pat. No. 4,938,763 to Dunn et al., U.S. Pat. No. 5,747,058 to Tipton and Holl and U.S. Pat. No. 5,488,075 to Guha.

After administration the solvent carrier disperses into the subject leaving the implant material behind. A problem is that the solvents employed in these methods have needed to be aggressive in order to dissolve the implant material. Aggressive solvents then become a part of the problem as the subject is injected with potentially toxic materials that may cause further illness or ailment.

A further alternative method considered is the use of thermo-reversible gels. These gels solidify in warm conditions—opposite to standard gels which solidify on cooling. By injecting the cold, usually room temperature, thermo-reversible gel, the gel solidifies in the subject (assuming the subject is warm blooded) and hence the implant forms. A typical example is described in U.S. Pat. No. 5,702,717 to Cha et al.

The above prior art relates to use in humans and animals, particularly livestock. An alternative for depot use is in plants, especially woody plants. Typically, treatment of trees is done via the roots e.g. liquid fertilisers. One technique used for treatment of fungus disease in a tree is to insert an impregnated dowel into the tree trunk itself. The dowel is impregnated with fungicide which is taken up by the sap of the tree. This method however involves damage to the tree i.e. the drilling of a hole. In addition the active agent is only able to be applied to the outside of the dowel and is essentially a one off release i.e. there is no sustained release of active which can be required to treat certain plant diseases.

It is therefore an object of the present invention to provide a formulation for easy and cost effective administration of a biodegradable implant that utilizes materials that are pharmaceutically and physiologically acceptable and effective.

It is a further object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### SUMMARY OF THE INVENTION

According to one aspect of the present invention there is provided a formulation for parenteral administration to a subject, including:
at least one water miscible solvent;
at least one gelling agent;
at least one active agent;
characterized in that the gelling agent is in particulate form and suspended in the solvent.

According to a further aspect of the present invention there is provided a formulation as described above wherein, when the formulation is administered to a subject, a depot forms which contains the active agent. Preferably, this agent is then released from the depot on a sustained basis. Preferably, the sustained basis is selected from: over a time period of days; a steady rate of release; and combinations thereof.

The composition of the present invention is such that on entry into the subject, the solvent disperses and the gelling agent coagulates to form a depot within the subject and from which the active agent is released on a sustained basis.

This has clear advantages in terms of the fact that a standard injection syringe can be used to administer the depot, and, due to the natural depot forming characteristics of the formulation, the formulation can be administered to areas of a subject not easily reached using prior art methods including oral administration, insertion into a cavity or surgery.

It is understood by the Applicant that the formulation of the gelling agent at least physically contains the active agent from dissolution into the subject on initial administration.

Preferably the formulation is administered parenterally, where, for the purposes of this specification, ‘parenterally’ is defined as by injection via a route selected from the group consisting of: subcutaneous; intramuscular; intraorbital; intracapsular; intraspinal; intrasternal; intravenous; within a plant; and other such known routes. More preferably the route used is selected from the group consisting of: subcutaneous; intramuscular; and combinations thereof.

The subject to which this formulation can be applied includes animals, (including humans) and plants.

The preferred embodiment is for administration to an animal. Preferably, the animals are livestock selected from the group consisting of: cattle; sheep; deer.

This type of animal is preferred as the high value of the animal justifies the cost of the treatment. In addition, it is both undesirable from a cost point of view and often difficult to administer a number or treatments to livestock as they may often be in remote locations and distressed by such treatments. By being able to inject the animal, for example once a season, the cost and labour of animal management is thus reduced.

A preferable group of plants to which this formulation may be used is woody plants. Most preferably, the plants are trees. In this embodiment, it is envisaged that by way of example, a fungicide is used as the active agent and the formulation is injected into the tree, for example into a small hole drilled into the tree. The aqueous environment inside the tree (e.g. sap) performs a similar function to the blood stream in animals. It will be appreciated that other plant active agents may also be used in this application.

For the purposes of the invention, a water miscible solvent is defined as a liquid that is susceptible of being dissolved in, or mixing with, water. This is important in order to ensure that the solvent used does not linger in the subject and cause possible side effects. A useful point of the invention is that often, water miscible solvents are less aggressive solvents and hence more physiologically acceptable.

In preferred embodiments, the solvent is selected from the group consisting of: ethanol; glycerin; polyethylene glycol, propylene glycol and combinations thereof.

It is the Applicant's understanding that the gelling agent forms a gel or semi-solid on dispersal of the solvent. For the purpose of this specification, ‘gel or semi-solid’ is defined as a viscous or solid like material formed by the aid of a suitable gelling agent. Such gels have a moderate to high viscosity—that is they do not pour freely. In preferred embodiments, gel materials are selected from the group consisting of: cyclodextrins; polyethylene oxide; hydroxypropyl methylcellulose; polyvinyl alcohol; polyvinyl pyrrolidone; methylcellulose; ethylcellulose; hydroxyethyl cellulose; sodium carboxymethyl cellulose; dextran; gelatine; pectin; sodium poly(acrylic acid); carboxymethyl cellulose and combinations thereof.

These gels are preferred due to their pharmaceutical and physiological acceptability and ability to form gels of desirable viscosity for use in such depot formation.

For embodiments administered to cattle, the preferred formulation includes 5 to 15% w/w polyethylene oxide, 10 to 50% w/w ethanol, 30 to 90% w/w glycerin and active agent quantities as required for the application.

It is the understanding of the Applicant that the formulation can be used with any active agent. The agent may have biological activity, chemical activity, physical activity, and combinations thereof. Most preferably the agent is biologically active.

Typical agents include those selected from the group consisting of: antibacterial agent; antifungal agent; anti-inflammatory agent; antiparasitic agent; antineoplastic agent; analgesic agent; anaesthetic agent; antipsychotic agent; vaccine; central nervous system agent; growth factor; hormone; antihistamine; osteoinductive agent; cardiovascular agent; anti-ulcer agent; bronchodilating agent; vasodilating agent; birth control agent; antihypertensive agent; anticoagulant; antispasmodic agent; fertility-enhancing agent; and combinations thereof. For plant applications, agents are preferably fungicides.

It will be appreciated by those skilled in the art that, as the formulation is substantially inert with respect to the active agent, any type of active agent can be included. Preferably though, the active agent is in the form of a solid, a liquid or combinations thereof e.g. a solid suspended in a liquid.

The agent is dispersed in either: the gelling agent; the solvent; and combinations thereof.

A plurality of agents may be used and may be incorporated entirely within the gelling agent, the solvent or a mixture in both the solvent and gelling agent. In preferred embodiments, the agent is evenly distributed within the solvent and/or gelling agent.

In preferred embodiments, the gelling agent is distributed evenly throughout the solvent as a particulate suspension. This has the advantage of even dispersion when injected into the subject.

Particulates are of a size range from 1 nm to 5 mm in diameter. More preferably the diameter is from 1 nm to 0.1 mm in diameter.

The suspension remains largely stable (biologically, chemically and physically) when stored. Settling can occur. Simple shaking of the formulation e.g. by hand, will re-establish the desired particulate suspension.

It is the Applicant's understanding that when the formulation is administered to the subject, two key mechanisms occur. Firstly, the solvent disperses naturally into the subject fluids. This is a result of the water miscible solvent being introduced to an aqueous environment e.g. an animal's body. For example, in the case of an animal, the solvent dissolves into the animal's blood stream. The solvent, being water miscible, easily dissolves into such environments without leaving harmful residue levels.

Secondly, on dispersal of the liquid in which the particles are suspended (the solvent), the gelling agent particles coagulate to form a depot.

For the purposes of this specification, ‘coagulation’ is defined as the dissolution of a particulate material into a gelatinous mass which may also be called clotting, clumping or combining.

It is the Applicant's understanding that the depot formed, traps the active agent within the depot in a relatively short period of time such that any free active agent (e.g. agent in the solvent solution) is substantially captured by the coagulating process before being carried away from the depot.

For the purposes of this specification ‘depot’ is defined as a substance (preferably containing an active agent) that is retained in close proximity to the site of injection so that release of the active agent occurs over a prolonged period of time.

In preferred embodiments, the depot formed is of a consistency selected from the group consisting of: a viscous material; a gel or semi-solid; and combinations thereof.

In preferred embodiments, the depot remains subcutaneous if the depot is administered subcutaneous or intramuscular if the depot is administered intramuscular.

In preferred embodiments, the depot formed is water soluble. It is understood by the applicant that the depot erodes/dissolves in the solution environment of the subject over time and in doing so releases the active agent into the subject.

Most preferably, dissolution of the depot occurs over a sustained and/or regular length of time.

Typical rates of release are dependent on the active agent desired dosage. The rate of release varies according to factors selected from the group consisting of: initial particle size; levels of gel in the formulation; the amount and type of active agent; levels of any additional materials in the formulation; the subject; subject metabolism; the administration site; and combinations thereof.

In preferred embodiments, the ingredients used are pharmaceutically and physiologically acceptable.

In further embodiments, other pharmaceutically and physiologically acceptable agents can be included that are substantially inert with respect to the active agent or agents and mechanisms of the formulation as described above.

A further advantage of the present invention is that leakage from the injection site is minimized or removed altogether. As detailed above, the gelling characteristics of the formulation bind the active agent within close proximity of the injection site. This avoids back flow of formulation out through the injection point thus stopping unwanted waste of agent and also gives a clean wound/administration area.

According to a further aspect of the present invention, there is provided a method of treatment of a subject requiring such treatment, by parenteral administration of a formulation to a subject, wherein the formulation includes:
at least one water miscible solvent;
at least one gelling agent;
at least one active agent;
characterized in that the gelling agent is in particulate form and suspended in the solvent.

According to a yet further aspect of the present invention, there is provided the use of a formulation for treatment of a subject requiring such treatment, by parenteral administration of a formulation to a subject, wherein the formulation includes:
at least one water miscible solvent;
at least one gelling agent;
at least one active agent;
characterized in that the gelling agent is in particulate form and suspended in the solvent.

It can be seen from the above description that there is provided a formulation capable of being injected into a subject such as an animal that forms a sustainable release depot.

The present invention uses pharmaceutically and physiologically acceptable materials that can be easily injected whilst still providing an implant for sustained release of an active agent. By use of the depot formulation of the present invention, it is easier to dose the subject as only one injection is required rather than the difficulties of surgery or a series of treatments associated with tablets or liquids. In addition, as the formulation uses compounds that are biologically ‘friendly’, harmful residues are avoided such as those from aggressive solvents.

### BRIEF DESCRIPTION OF THE DRAWINGS

Further aspects of the present invention will become apparent from the ensuing description which is given by way of example only and with reference to the accompanying drawings in which.

FIG. 1 shows the exterior of a portion of excised deer muscle tissue comparing a control sample against that of the present invention; and,

FIG. 2 shows the interior of a portion of excised deer muscle tissue comparing a control sample against that of the present invention; and,

FIG. 3 shows the interior of a portion of teat cistern of excise bovine mammary gland showing the result from an infusion of formulation of the present invention.

### DETAILED DESCRIPTION OF THE PREFERRED EMBODIMENT

The invention relates to a formulation consisting of a gelling agent or agents, a water miscible solvent or solvents and an active agent or agents. The aim of the suspension is to deliver active agent or agents to a subject in the form of a depot that, on parenteral administration to the subject, forms a depot. The depot will disperse in time and during this time period, active agent or agents are released into the subject.

The following examples illustrate the gel forming characteristics of the formulation.

### EXAMPLE 1

The nature of the depot, i.e. viscous solution, gel or semi-solid is described by addition of gelling agent to solvent to show that the effect of selection of depot formation material on the final depot characteristics. Results are shown in Table 1 below.

TABLE 1
Materials Used and the Depot Characteristics

| Gelling Agent | Solvent | Characteristics |
|---|---|---|
| Polyethylene oxide | Ethanol | Viscous depot |
| Polyethylene oxide | Ethanol/Glycerin | Viscous depot |
| Hydroxypropyl β- | Ethanol | Viscous depot |
| cyclodextrin | | |

### EXAMPLE 2

By way of further illustration, the experiment in Example 1 is repeated using polyethylene oxide as the gelling agent and ethanol as the solvent.
Polyethylene oxide (1 g) was suspended in ethanol (9 g).
0.1 mL of this suspension was dispensed from a syringe onto a drop of water (approximately 0.05 mL) at the bottom of a beaker.
A gel was observed to rapidly form which held its shape and could not be poured from the beaker.

### EXAMPLE 3

Animal trials were conducted to determine the depot forming ability of the formulation.

A formulation of the present invention is used with polyethylene oxide used as the gelling agent and a combination of ethanol and glycerin as the solvents. Sodium fluoresceine is used as a dye to show experimental results.

A control sample is used for comparison which includes no gelling agent (only glycerin, ethanol and sodium fluoresceine).

Tests were completed on deer muscle by injection of both formulations (invention and control) into the muscle tissue.

The composition of each formulation is as shown in Table 2 below.

TABLE 2
Formulations Used

| Compound | Test formulation | | Control formulation | |
|---|---|---|---|---|
| Polyethylene oxide | 10% | w/w | | |
| Ethanol | 45% | w/w | 50% | w/w |
| Glycerin | 45% | w/w | 50% | w/w |
| Fluoresceine Na | 1 | mg/mL | 1 | mg/mL |

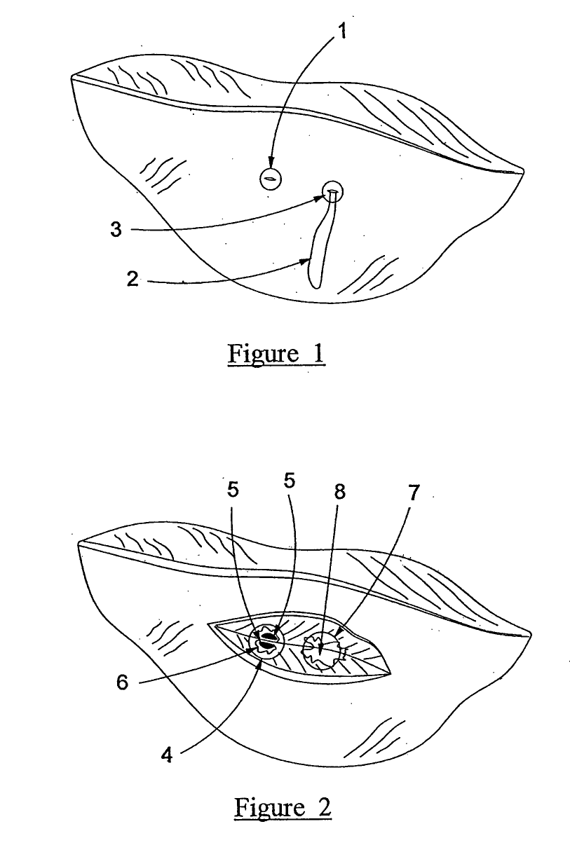

Referring to FIG. 1, the exterior results of injection of test and control formulations into excised deer muscle tissue is shown. The injection point of the test formulation (1) shows very little leakage of the formulation via the needle tract. The control formulation shows extensive leakage (2) via the needle tract (3).

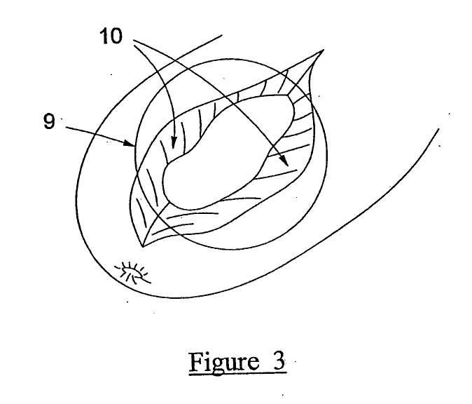

Referring to FIG. 2, the injection site of the tissue of FIG. 1 is shown. The injection site of the test formulation (4) shows the formed depot (5) and little distribution of dye into the surrounding tissue (6), whereas the control formulation (7) shows no depot has formed and extensive distribution of dye (8) into the surrounding tissue.

### EXAMPLE 4

The same invention formulation as per table 1 Example 3, is used on the teat cistern of bovine mammary gland.

As shown in FIG. 3, the invention formulation generally indicated by arrow (9) has gelled and adhered to the internal teat cistern wall (10).

Aspects of the present invention have been described by way of example only and it should be appreciated that modifications and additions may be made thereto without departing from the scope thereof as defined in the appended claims.

1. A formulation for parenteral administration to a subject, comprising:
at least one water miscible solvent;
at least one gelling agent; and
at least one active agent;
characterized in that the gelling agent is in particulate form and suspended in the solvent.

2. The formulation of claim 1, wherein on administration to a subject, the formulation forms a depot which contains the active agent.

3. The formulation of claim 1, wherein, on administration, the gelling agent coagulates around active agent to form the depot.

4. The formulation of claim 1, wherein the agent is released from the depot on a sustained basis.

5. The formulation of claim 4, wherein the sustained basis is selected from: over a time period of days, a steady rate of release, and combinations thereof.

6. The formulation of claim 1, wherein the gelling agent at least physically contains the active agent from dissolution into the subject on initial administration.

7. The formulation of claim 1, wherein the parenteral route is selected from the group consisting of: subcutaneous, intramuscular, intraorbital, intracapsular, intraspinal, intrastemal, intravenous, and within a plant.

8. The formulation claim 1, wherein the parenteral route used is selected from the group consisting of: subcutaneous, intramuscular, and combinations thereof.

9. The formulation of claim 1, wherein the subject is an animal.

10. The formulation of claim 9, wherein the animal is selected from the group consisting of: cattle, sheep, and deer.

11. The formulation of claim 10 where cattle are the animal and the formulation includes: 5 to 15% w/w polyethylene oxide; 30 to 50% w/w ethanol; 30 to 50% w/w glycerin; active agent quantities as required for the application.

12. The formulation of claim 1, wherein the subject is a plant.

13. The formulation of claim 12, wherein the plant is a woody plant.

14. The formulation of claim 12, wherein the plant is a tree.

15. The formulation of claim 12, wherein the active agent is a fungicide and the formulation is injected to fill a hole in the tree.

16. The formulation of claim 1, wherein the solvent is selected from the group consisting of: ethanol, glycerin, and combinations thereof.

17. The formulation of claim 1, wherein the gelling agent is selected from the group consisting of: alginates, cyclodextrins, polyethylene oxide, polylysine, and combinations thereof.

18. The formulation of claim 1, wherein the agent is biologically active.

19. The formulation of claim 1, wherein active agents include those selected from the group consisting of: antibacterial agent, antifungal agent, fungicides, anti-inflammatory agent, antiparasitic agent, anti-neoplastic agent, analgesic agent, anaesthetic agent, antipsychotic agent, vaccine, central nervous system agent, growth factor, hormone, antihistamine, osteoinductive agent, cardiovascular agent, antiulcer agent, bronchodilating agent, vasodilating agent, birth control agent, antihypertensive agent, anticoagulant, antispasmodic agent, fertility-enhancing agent, and combinations thereof.

20. The formulation of claim 1, wherein the active agent is in the form of a solid, a liquid; and combinations thereof.

21. The formulation of claim 1, wherein the agent is dispersed in: the gelling agent, the solvent, and combinations thereof.

22. The formulation of claim 21, wherein the agent is evenly distributed within: the solvent, the gelling agent, or both the solvent and gelling agent.

23. The formulation claim 1, wherein the gelling agent is distributed evenly throughout the solvent as a particulate suspension.

24. The formulation of claim 1, wherein particulates are of a size range from 1 nm to 5 mm in diameter.

25. The formulation of claim 1, wherein particulates are of a size range from 1 nm to 0.1 mm in diameter.

26. The formulation claim 1, wherein the depot forms within in a relatively short period of time such that any free active agent is substantially captured before being carried away from the depot.

27. The formulation of claim 1, wherein the depot formed is of a consistency selected from the group consisting of: a viscous material, a gel or semi-solid, and combinations thereof.

28. The formulation of claim 27 wherein the depot formed is water soluble.

29. The formulation of claim 1, wherein the rate of release varies according to factors selected from the group consisting of: initial particle size, levels of gel in the formulation, the amount and type of active agent, levels of any additional materials in the formulation, the subject, subject metabolism, the administration site, and combinations thereof.

30. The formulation of claim 1, wherein other pharmaceutically and physiologically acceptable agents are included that are substantially inert with respect to the active agent or agents.

31. A method of treatment of a subject requiring such treatment, comprising administering parenterally a formulation of claim 1.
